(19) United States
(12) Patent Application Publication
(10) Pub. No.: US 2015/0031101 A1
(43) Pub. Date: Jan. 29, 2015

(54) BACTERIAL CELL HAVING ENHANCED SUCCINIC ACID PRODUCTION AND A METHOD FOR PRODUCING THE SUCCINIC ACID USING THE SAME

(71) Applicant: Samsung Electronics Co., Ltd., Suwon-si (KR)

(72) Inventors: Joonsong PARK, Seoul (KR); Jinhwan Park, Suwon-si (KR); Soonchun Chung, Seoul (KR); Jiae Yun, Hwaseong-si (KR); Kwangmyung Cho, Seongnam-si (KR)

(21) Appl. No.: 14/444,749

(22) Filed: Jul. 28, 2014

(30) Foreign Application Priority Data
Jul. 26, 2013 (KR) 10-2013-0088972

(51) Int. Cl. C12P 7/46; C12N 15/77

(52) U.S. Cl. CPC C12P 7/46; C12N 15/77
USPC 435/145; 435/252.32; 435/252.3

(57) ABSTRACT

A genetically engineered bacterial cell wherein activity of a pathway in the cell of converting α-ketoglutarate into succinate semialdehyde; or activity of succinyl semialdehyde dehydrogenase in the cell is increased compared to the activity in a non-genetically engineered cell of the same type, and a method of producing succinic acid by using the same.

### CROSS-REFERENCE TO RELATED APPLICATIONS

This application claims the benefit of Korean Patent Application No. 10-2013-0088972, filed on Jul. 26, 2013, in the Korean Intellectual Property Office, the disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 92,365 bytes ASCII (Text) file named “718195_ST25.TXT,” created Jul. 28, 2014.

### BACKGROUND

1. Field

The present disclosure relates to a genetically modified bacterial cell having enhanced succinic acid production and a method for producing succinic acid by using the same.

2. Description of the Related Art

Corynebacterium is a gram positive bacteria used for the production of amino acids such as glutamate, lysine, and threonine. Corynebacterium has advantages as an industrial strain because it can grow in a variety of conditions and environments, its genome structure is stable, and poses little risk as an environmental hazard. Corynebacterium glutamicum is an aerobic bacteria. In anaerobic conditions, the metabolic processes of Corynebacterium glutamicum other than those necessary to produce minimum energy for survival cease. As a result, lactic acid, acetic acid, and succinic acid. are produced and released from Corynebacterium glutamicum.

Tricarboxylic acid (TCA) cycle is a metabolic process utilized by organisms such as Corynebacterium to produce energy and intermediate products. The intermediates produced in the TCA cycle are converted into useful chemical materials via various metabolic processes in cells. Succinic acid is a kind of dicarboxylic acid used as a raw material for the manufacture of biodegradable polymers, medicines, foods and cosmetics. Most industrially used succinic acid is synthesized from n-butane and acetylene derived from crude oil or liquefied natural gas. Only a small amount of succinic acid used for special purposes such as medicines and foods has been produced by fermentation processes using a microorganism. A chemical synthesis process generally generates a large amount of hazardous materials, and uses fossil fuels, which are rapidly depleted. Thus, it is exists a need to develop a method for efficiently producing succinic acid using microorganisms.

### SUMMARY

Provided is a genetically engineered bacterial cell wherein activity of a pathway in the cell of converting α-ketoglutarate (AKG) into succinate semialdehyde (SSA) or activity of succinyl semialdehyde dehydrogenase (SSADH) in the cell is increased as compared to the activity in a parent cell. The pathway of converting α-ketoglutarate (AKG) into SSA includes α-ketoglutarate decarboxylase (KGDC); glutamate:succinate semialdehyde transaminase and glutamate decarboxylase; or a combination thereof, wherein the KGDC catalyzes the conversion of α-ketoglutarate into SSA, and the SSADH catalyzes the conversion of SSA into succinic acid. Thus, in one aspect, the bacterial cell is engineered to increase the activity of succinyl semialdehyde dehydrogenase (SSADH), α-ketoglutarate decarboxylase (KGDC), glutamate:succinate semialdehyde transaminase, glutamate decarboxylase, or a combination thereof, in the genetically engineered bacterial cell as compared to a parent cell. According to another aspect of the present invention, provided is a genetically engineered bacterial cell in which the copy number of a gene encoding at least one selected from the group consisting of a pathway of converting α-ketoglutarate into SSA; and succinyl semialdehyde dehydrogenase is increased, or the expression of the gene(s) is increased, as compared to a parent cell. In a related aspect, the engineered bacterial cell comprises an increased copy number of a gene encoding succinyl semialdehyde dehydrogenase (SSADH), α-ketoglutarate decarboxylase (KGDC), glutamate:succinate semialdehyde transaminase, glutamate decarboxylase, or a combination thereof, as compared to a parent cell.

Further provided is a method for producing succinic acid by using the genetically engineered bacterial cell. The method comprises culturing the genetically engineered bacterial cell, whereby the cell produces succinic acid; and recovering succinic acid from the culture.

Also provided is a method of improving succinic acid production by a bacterial cell, the method comprising increasing the activity of succinyl semialdehyde dehydrogenase (SSADH), α-ketoglutarate decarboxylase (KGDC), glutamate:succinate semialdehyde transaminase, and glutamate decarboxylase, or a combination thereof, in the cell. In one aspect, the activity of one or more of the enzymes is increased by increasing the copy number of one or more genes encoding the one or more enzymes.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings of which:

FIG. 1 is an expression vector map of a pGSK+ vector;

FIG. 2 is an expression vector a map of pGS-Term vector;

FIG. 3 is an expression vector map of pGS-EX4 vector; and

FIG. 4 is an expression vector map of a vector used for the introduction of a gene encoding α-ketoglutarate decarboxylase and a gene encoding succinyl semialdehyde dehydrogenase into a microorganism.

### DETAILED DESCRIPTION

An aspect of the present invention provides a genetically engineered bacterial cell, wherein activity of a pathway in the cell of converting α-ketoglutarate (AKG) into succinate semialdehyde (SSA) or activity of succinyl semialdehyde dehydrogenase (SSADH) in the cell is increased as compared to the activity in a parent cell. Another embodiment provides a genetically engineered bacterial cell, wherein the activity of α-ketoglutarate decarboxylase (KGDC); glutamate: succinate semialdehyde transaminase and glutamate decarboxylase; or a combination thereof which catalyzes the conversion of α-ketoglutarate into SSA, is increased as compared to a parent cell. The glutamate: succinate semialdehyde transaminase may catalyze the following chemical reaction: 4-aminobutanoate+2-oxoglutarate→succinate semialdehye+L-glutamate.

The glutamate: succinate semialdehyde transaminase may be an enzyme included in EC 2.6.1.19. The glutamate: succinate semialdehyde transaminase may be an Escherichia coli-derived gabT (SEQ ID NO: 67) or an enzyme derived from S. cerevisiae. The glutamate decarboxylase may convert glutamate into CO₂ and 4-aminobutyrate. The glutamate decarboxylase may be included in EC 4.1.1.15. The glutamate decarboxylase may be an Escherichia coli-derived glutamate decarboxylase alpha (SEQ ID NO: 68) or an Escherichia coli-derived glutamate decarboxylase beta (SEQ ID NO: 69), or an enzyme derived from S. cerevisiae.

As used herein the term “increase in activity” or “increased activity” of a cell, pathway, enzyme, or polypeptide may refer to a detectable increase in the activity thereof. “Increase in activity” or “increased activity” may also refer to an activity level of a modified (e.g., genetically engineered) cell, pathway, protein, or enzyme that is higher than that of a comparable cell, pathway, protein, or enzyme of the same type, such as a cell, pathway, protein, or enzyme that does not have a given modification (e.g, the original or parent or “wild-type” cell, pathway, protein, or enzyme). The comparable cell may be an unmodified parent cell lacking a specific genetic modification that produced the genetically engineered cell. For example the activity of the cell, pathway, enzyme, or polypeptide may have activity level that is increased by about 5%, about 10%, about 15%, about 20%, about 30%, about 50%, about 60%, about 70%, or about 100% in comparison with the same activity of the unmodified cell, pathway, enzyme, or polypeptide. An activity of polypeptide may be measured by using a method known to those of ordinary skill in the art. “Activity increase” or “increased activity” of a pathway may be caused by an increase of the activity an enzyme or a polypeptide related to the pathway.

The activity increase of the enzyme or peptide may be achieved by increased expression of the gene encoding the enzyme or peptide. The expression increase may be caused by introduction of a polynucleotide encoding the polypeptide, by increase of the number of copies of the polypeptide, or by mutation of a regulatory region of the polynucleotide. A polynucleotide which is introduced externally or whose copy number is increased may be endogenous or exogenous to the cell. The endogenous gene refers to a gene which has existed on a genetic material included in a microorganism. The exogenous gene refers to a gene which is introduced to a host cell by a method such as integration to a host cell genome. An introduced gene may be homologous or heterologous the host cell.

The term used herein “increase of activity” or “increased activity” of a pathway, an enzyme, or a polypeptide also includes causing by genetic engineering a cell to have an activity which is not native or preexisting in the cell.

The term “copy number increase,” as used herein, may refer to an increase of copy number by the introduction of a gene or amplification of a gene, and may be achieved by genetically engineering a cell to have a gene that does not naturally exist in the cell. The introduction of a gene may be mediated by a vehicle such as a vector. The introduction may be a transient introduction in which the gene is not integrated to a genome, or the gene may be inserted into the cellular genome. The introduction may be performed, for example, by introducing a vector into the cell, wherein the vector comprises a polynucleotide encoding a target polypeptide, and expressing the vector in the cell separately from the genome, or by integrating the polynucleotide into the genome.

The term “gene” refers to a nucleic acid fragment expressing a specific protein and may include a coding region as well as regulatory sequences such as a 5′-non coding sequence or a 3′-non coding sequence.

The term “heterologous” means “foreign,” or “not native.” The term “homologous” means “native.”

The term “secretion” means transport of a material from the inside of a cell to a periplasmic space or an extracellular environment.

The terms “cell,” “strain,” or “microorganism” may be used interchangeably and include bacteria.

The term used herein “activity decrease” or “decreased activity” of an cell, pathway, enzyme or polypeptide (e.g., a modified or genetically engineered cell, pathway, enzyme, or polypeptide) refers to an activity level that is lower than the activity level measured in a comparable cell, pathway, enzyme, or polypeptide (e.g., a cell, pathway, enzyme, or polypeptide of the same kind that does not contain the modification or genetic engineering), or that the activity is absent. For example, the activity of a modified or genetically engineered cell, pathway, enzyme, or polypeptide may be decreased by about 20%, about 30%, about 40%, about 50%, about 55%, about 60%, about 70%, about 75%, about 80%, about 85%, about 90%, about 95%, or about 100% in comparison with to an unmodified cell, pathway, enzyme, or polypeptide of the same type. Decreased enzyme activity may be verified by using a method known to those of ordinary skill in the art for measuring an enzyme activity. The decrease in activity may be caused by expression of an enzyme that is mutated so as to exhibit no activity or decreased activity compared to the activity level of an unmodified enzyme (e.g., enzyme produced by an unmodified cell), or by the under-expression (or lack of expression) of a gene encoding an enzyme as compared to the expression of an unmodified gene of the same type (e.g., expression of the same gene in an unmodified cell).

The decrease of enzyme activity may be caused by deletion or disruption of a gene encoding the enzyme. The term “deletion” or “disruption” refers to mutating, substituting, or deleting a part of a gene or an entire gene, or a part of a regulatory element or an entire regulatory element such as a prompter region and a terminator region of the gene or inserting at least one base to the gene so that the gene may not be expressed, or the amount of expression may be decreased, or the enzyme activity may not be shown or decreased even when the gene is expressed. The deletion or disruption of a gene may be achieved through genetic engineering such as homologous recombination, mutation, or molecular evolution. When a cell includes multiple copies of a same gene or two or more paralog genes of different polypeptides, one or more genes may be deleted or disrupted.

The term “sequence identity” of a nucleic acid or a polypeptide used herein refers to a degree of similarity of base groups or amino acid residues between two aligned sequences, when the two sequences are aligned to match each other to the greatest extent possible, at corresponding positions. The sequence identity is a value that is measured by aligning to an optimum state and comparing the two sequences at a particular region of interest, wherein a part of the sequence within the particular modified cell may be added or deleted compared to a reference sequence. A sequence identity percentage may be calculated, for example, by 1) comparing the two sequences aligned within the whole comparing region to an optimum 2) obtaining the number of matched locations by determining the number of locations represented by the same amino acids of nucleic acids in both of the sequences, 3) dividing the number of the matched locations by the total number of the locations within the comparing region (i.e., a range size), and 4) obtaining a percentage of the sequence identity by multiplying 100 to the result. The sequence identity percent may be determined by using a common sequence comparing program, for example, BLASTN (NCBI), CLC Main Workbench (CLC bio), MegAlign™ (DNASTAR Inc).

In confirming that different polypeptides or polynucleotides have the same or similar function or activity, sequence identities may be used. For example, the sequence identities may include about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, about 99% or greater, or 100%.

The cell may have a succinic acid-producing ability. The cells may have an increase in succinic acid-producing ability as compared to a parent cell (e.g., non-genetically engineered cells), by being genetically engineered such that the activity of a pathway of converting α-ketoglutarate into SSA; and/or succinyl semialdehyde dehydrogenase in the cell is increased compared to a parent cell (e.g., non-genetically engineered cell).

The cells may be capable of producing succinic acid in aerobic, anaerobic or microaerobic conditions. The microaerobic condition may refer to a condition where oxygen of a level lower than the atmospheric oxygen concentration is dissolved into a culture medium. The low level of oxygen refers to from about 0.1% to about 10%, about 1% to about 9%, about 2% to about 8%, about 3% to about 7%, or about 4% to about 6% of the atmospheric oxygen concentration. The cells may produce succinic acid via oxidative TCA cycle as well as via reductive TCA cycle. The genetically engineered cells may have an increase in succinic acid-producing ability as compared to the non-genetically engineered cells by producing succinic acid via oxidative TCA cycle. The genetically engineered cells may have an increase in succinic acid-producing ability as compared to the non-genetically engineered cells by producing succinic acid for example, in a microaerobic condition via oxidative TCA cycle.

The cell may be Corynebacterium. The cells may be, for example, Corynebacterium glutamicum, Corynebacterium thermoaminogenes, Brevibacterium flavum or Brevibacterium lactofermentum.

The activity may be increased by an increase in the copy number of at least one gene encoding a polypeptide used in the pathway of converting α-ketoglutarate into SSA, and/or an increase in the copy number of a gene encoding succinyl semialdehyde dehydrogenase. An “increase” in copy number of the genetically engineered cell as compared to an unmodified “parent” cell includes, for instance, introduction of a gene into the cell that does not exist in the unmodified “parent” cell. The gene encoding a polypeptide used in the pathway of converting α-ketoglutarate into SSA may include a gene encoding KGDC; a gene encoding glutamate: succinate semialdehyde transaminase and a gene encoding glutamate decarboxylase; or a combination thereof which catalyzes the conversion of α-ketoglutarate into SSA. The increase in copy number may be due to the introduction or amplification of the gene. The introduction may be mediated by a vehicle such as a vector. The introduction may be transient where the gene is not integrated into its chromosome or the gene may be inserted into its chromosome. The introduction may be achieved by introducing a vector where a polynucleotide encoding KGDC or succinyl semialdehyde dehydrogenase is inserted into a cell, and replicating the vector in the cell or integrating the polynucleotide into the chromosome. The gene may include a regulatory sequence in addition to a nucleotide sequence encoding a protein. The gene may be endogenous or exogenous.

In addition, the activity may be increased by modification of regulatory sequences for expressing the gene(s). The regulatory sequence may be a promoter sequence for expressing the gene or a transcription terminator sequence. The promoter may be P₁₈₀ promoter, or P₄₅ promoter. The transcription terminator may be rrnBT terminator from an E. coli. The modification may a replacement of the endogenous promoter or terminator with the exogenous promoter or terminator. The regulatory sequence may also be a nucleotide sequence encoding a motif which can influence the expression of the gene. The motif may be, for example, a secondary structure-stabilizing motif, an RNA destabilizing motif, a splice-activating motif, a polyadenylation motif, an adenine-rich sequence, or an endonuclease recognition site.

In addition, the activity may be increased by mutation of a polypeptide and/or gene encoding a polypeptide that changes the amino acid sequence of at least one polypeptide used in a pathway of converting α-ketoglutarate into SSA; and/or succinyl semialdehyde dehydrogenase. The modification may be produced by deletion, insertion, substitution of a nucleotide sequence in a protein encoding region, or combinations thereof. The modification may, for example, increase the affinity of an enzyme included in the pathway of converting α-ketoglutarate into SSA or succinyl semialdehyde dehydrogenase for its substrate. Accordingly, the modification may increase the specific activity of the enzyme itself.

The KGDC may be an enzyme classified to EC.4.1.1.71. For example, the KGDC is derived from Corynebacterium., Rhodococcus sp., Mycobacterium sp., or Escherichia sp. The gene encoding the KGDC may be derived from, for example, Corynebacterium glutamicum, Corynebacterium callunae, Corynebacterium efficiens, Corynebacterium ulcerans, Corynebacterium halotolerans, Corynebacterium pseudotuberculosis, Corynebacterium durum, Corynebacterium striatum, Rhodococcus pyridinivorans, Rhodococcus ruber, Mycobacterium abscessus, Mycobacterium smegmatis, Escherichia coli, Escherichia fergusonii, or combinations thereof. The gene encoding the KGDC may be, for example, a polynucleotide encoding an amino acid sequence having a sequence identity of 95% or higher with the amino acid sequence of SEQ ID NO:1 or SEQ ID NO: 2. The gene encoding the KGDC may have, for example, a nucleotide sequence having a sequence identity of 95% or higher with the nucleotide sequence of SEQ ID NO: 3 or SEQ ID NO: 4.

The succinyl semialdehyde dehydrogenase (SSADH) may be an enzyme classified to EC.1.2.1.24, EC.1.2.1.79, or EC.1.2.1.16. The SSADH may be dependent on NAD+−, NADP+, or both. The SSADH may be CoA-independent. For example, the SSADH may be derived from Corynebacterium., Rhodococcus sp., Gordonia sp., Mycobacterium sp., or Escherichia sp. The SSADH may be an Escherichia coli-derived gabD1, gabD2, or gabD3. For example, the gene encoding the SSADH may be a polynucleotide encoding an amino acid sequence having a sequence identity of 95% or higher with an amino acid sequence of SEQ ID NOS: 5 (gabD2), 63 (gabD1), and 64 (gabD3). The gene encoding SSADH may have a nucleotide sequence having a sequence identity of 95% or higher with a polynucleotide of SEQ ID NOS: 6 (gabD2), 65 (gabD1), and 66 (gabD3).

The cell may be characterized in that the gene encoding succinyl-CoA synthetase (SucCD), which catalyzes the conversion of succinic acid into succinyl-CoA, is inactivated or attenuated. The succinyl-CoA synthetase may be classified as EC.6.2.1.5. The gene encoding succinyl-CoA synthetase may be, for example, a polynucleotide encoding an amino acid sequence having a sequence identity of 95% or higher with an amino acid sequence of SEQ ID NO:7 or SEQ ID NO: 8.

The cell may be characterized in that the gene encoding L-lactate dehydrogenase (LDH) which catalyzes the conversion of pyruvate into lactate, the gene encoding pyruvate oxidase (PoxB) which catalyzes the conversion of pyruvate into acetate, the gene encoding phosphotransacetylase (PTA) which catalyzes the conversion of acetyl-CoA into acetyl phosphate, the gene encoding acetate kinase (AckA) which catalyzes the conversion of acetyl phosphate into acetate, the gene encoding acetate CoA transferase (ActA) which catalyzes the conversion of acetate into acetyl-CoA, or a combination thereof is inactivated or attenuated. The LDH may be an enzyme classified as EC.1.1.1.27. The LDH may have, for example, an amino acid sequence having a sequence identity of 95% or higher with an amino acid sequence of SEQ ID NO:9. The PoxB may be an enzyme classified as EC.1.2.5.1. The PoxB may have, for example, an amino acid sequence having a sequence identity of 95% or higher with an amino acid sequence of SEQ ID NO: 10. The PTA may be an enzyme classified as EC.2.3.1.8. The PTA may have, for example, an amino acid sequence having a sequence identity of 95% or higher with an amino acid sequence of SEQ ID NO: 11. The AckA may be an enzyme classified as EC.2.7.2.1. The AckA may have, for example, an amino acid sequence having a sequence identity of 95% or higher with an amino acid sequence of SEQ ID NO:12. The ActA may be an enzyme classified as EC.2.8.3.9. The ActA may have, for example, an amino acid sequence having a sequence identity of 95% or higher with an amino acid sequence of SEQ ID NO:13. When the genes or combinations thereof are inactivated or attenuated, the activity of converting pyruvate into lactate, the activity of converting pyruvate into acetate, or combinations thereof in the cell may be lost or decreased.

The term, “inactivation” or “inactivated” used herein, may refer to a gene (or act of generating a gene) which cannot be expressed or encodes a protein having no activity when expressed. The term, “attenuation” or “attenuated” used herein, may refer to a gene (or act of generating a gene) whose expression level is reduced as compared to the expression level of the same gene in a non-engineered bacterial strain or a parental bacterial strain of the same type (e.g., a “wild-type” strain), or a gene which encodes a protein with decreased activity as compared to the activity of the same protein in a non-engineered bacterial strain or a parental bacterial strain of the same type (e.g., a “wild-type” strain). The inactivation or attenuation may be caused by a genetic modification, for example, substitution or addition mutation, or by deletion or all or part of the gene. Any mode of modification may be used, such as homologous recombination. The inactivation or attenuation may be achieved, for example, via transformation by inserting a vector including a partial nucleotide sequence of a gene by inserting into the cell, culturing the cell to induce a homologous recombination between the partial nucleotide sequence and the sequence of an endogenous gene, and selecting a cell where the homologous recombination occurred by a selective marker.

The cell may be one with increased activity of pyruvate carboxylase (PYC) which catalyzes the conversion of pyruvate into oxaloacetate. The increased activity may be achieved, for example, by the introduction of a gene encoding pyruvate carboxylase, which has an increased specific activity due to a mutation. The mutation may include substitution, addition, deletion, or combinations thereof. The substitution may be, for example, a substitution of the 458^{th} amino acid of SEQ ID NO: 14 from proline to serine.

According to another aspect of the present invention, provided is a genetically engineered bacterial cell, wherein the copy number of at least one selected from the group consisting of a gene encoding a polypeptide used in a pathway of converting α-ketoglutarate into SSA; and/or a gene encoding SSADH is increased, or the expression of the gene(s) is increased as compared to a non-genetically engineered cell, wherein the pathway of converting α-ketoglutarate into SSA includes KGDC; glutamate: succinate semialdehyde transaminase and glutamate decarboxylase; or a combination thereof which catalyzes the conversion of α-ketoglutarate into SSA, and the SSADH catalyzes the conversion of succinyl semialdehyde into succinic acid, is provided. The gene encoding the pathway of converting α-ketoglutarate into SSA may be a gene encoding KGDC; a gene encoding glutamate: succinate semialdehyde transaminase and a gene encoding glutamate decarboxylase; or a combination thereof which catalyzes the conversion of α-ketoglutarate into SSA.

The cell may be Corynebacterium. The cell may be, for example, Corynebacterium glutamicum, Corynebacterium thermoaminogenes, Brevibacterium flavum or Brevibacterium lactofermentum.

The copy number may be increased by introduction or amplification of the gene(s). The details of the introduction of the gene are the same as described above. The gene introduced may be endogenous or exogenous. The gene encoding the KGDC may be one encoding an enzyme classified as EC.4.1.1.71.

The gene encoding the KGDC may be derived from Corynebacterium. or Escherichia sp. including Escherichia coli. For example, the gene may be one encoding an amino acid sequence having a sequence identity of 95% or higher with an amino acid sequence of SEQ ID NO:1 or SEQ ID NO:2. The gene encoding the SSADH may be one encoding an enzyme classified as EC.1.2.1.16. The gene encoding the SSADH may be, for example, one derived from Corynebacterium sp., Rhodococcus sp., Gordonia sp., Mycobacterium sp., Anthrobacter sp., or Escherichia sp. For example, the gene may be one encoding an amino acid sequence having a sequence identity of 95% or higher with an amino acid sequence of SEQ ID NOS:5(gabD2), 63 (gabD1), and 64 (gabD3). The gene encoding the SSADH may have a nucleotide sequence having a sequence identity of 95% or higher with a polynucleotide of SEQ ID NOS: 6(gabD2), 65 (gabD1), and 66 (gabD3).

The expression of the gene(s) may be increased by modification of regulatory sequences for expressing the gene(s). The details of the regulatory sequence are the same as described above.

The cell may be one where a gene encoding succinyl-CoA synthetase is inactivated or attenuated. The cell may be one where L-lactate dehydrogenase gene, pyruvate oxidase gene, phosphotransacetylase gene, acetate kinase gene, acetate CoA transferase gene, or combinations thereof is inactivated or attenuated. The cell may be one with increased activity of pyruvate carboxylase which catalyzes the conversion of pyruvate into oxaloacetate. The details of such modifications are as described herein with respect to other embodiments.

According to further aspect of the present invention, provided is a method for producing succinic acid comprising culturing cell genetically modified cell as described above in a cell culture medium, whereby the cell produces succinic acid, and recovering succinic acid from the culture is provided. Other aspects of the genetically modified cell useful in this method are as described above with respect to other embodiments.

The culturing may be performed according to a suitable culture medium and conditions known in the art. A skilled person in the art can easily adjust the culture medium and conditions according to the microorganism to be selected. The culture method may include a batch culture, a continuous culture and a fed-batch culture.

The culture medium can include various carbon sources, nitrogen sources and a trace amount of components. The carbon sources may include, for example, a carbohydrate such as glucose, sucrose, lactose, fructose, maltose, starch, and cellulose; a fatty acid such as soybean oil, sunflowers oil, castor oil, coconut oil, palmitic acid, stearic acid, and linoleic acid; an alcohol such as glycerol and ethanol; an organic acid such as acetic acid; and/or combinations thereof. The culturing may be performed, for example, by using glucose as a carbon source. The nitrogen source may include, for example, an organic nitrogen source such as peptone, yeast extract, meat extract, malt extract, corn steep liquor (CSL), and soybean wheat; an inorganic nitrogen source such as urea, ammonium sulfate, ammonium chloride, ammonium phosphate, ammonium carbonate, and ammonium nitrate; and/or combinations thereof. The culture medium may include as a phosphorous source, for example, potassium dihydrogen phosphate, dipotassium hydrogen phosphate and its corresponding sodium-containing salt, a metal salt such as magnesium sulfate or iron sulfate. In addition, the culture medium may include amino acids, vitamins, and suitable precursors. The culture medium or individual components may be added to a culture broth in the form of a batch culture or a continuous culture.

Additionally, compounds such as ammonium hydroxide, potassium hydroxide, ammonia, phosphoric acid, and sulfuric acid may be added in an appropriate manner into a culturing broth during culture to adjust the pH of the culturing broth. Furthermore, an anti-foaming agent such as fatty acid polyglycol ester may be used to prevent generation of foams during culture.

The cell can be cultured in aerobic, microaerobic, or anaerobic conditions. The microaerobic condition refers to a culturing condition where a lower level of oxygen than that in the atmosphere is dissolved in a culture medium. The lower level of oxygen is, for example, from about 0.1% to about 10%, about 1% to about 9%, about 2% to about 8%, about 3% to about 7%, or about 4 to about 6%. Additionally, the microaerobic condition may be, for example, a culturing condition where the concentration of oxygen dissolved in a culture medium is from about 0.9 ppm to about 3.6 ppm, for example, from about 0.9 ppm to about 2.6 ppm, from about 0.9 ppm to about 1.6 ppm, from 1.0 ppm to about 3.6 ppm, from about 2.0 ppm to about 3.6 ppm, or from about 3.0 ppm to about 3.6 ppm. The culturing temperature may be, for example, from about 20° C. to about 45° C., or from about 25° C. to about 40° C. The cultivation may be continued until a desirable amount of target succinic acid is obtained.

The succinic acid can be recovered by using a conventional separation and purification method. The recovery can be also made by centrifugation, ion exchange chromatography, filtration, precipitation, or combinations thereof. For example, the culture can be centrifuged to remove biomass, and the resulting supernatant can be separated by ion exchange chromatography. P

Provided is a method of improving succinic acid production by a bacterial cell, the method comprising increasing the activity of succinyl semialdehyde dehydrogenase (SSADH), α-ketoglutarate decarboxylase (KGDC), glutamate:succinate semialdehyde transaminase, and glutamate decarboxylase, or a combination thereof, in the cell. Suitable means for increasing the activity of succinyl semialdehyde dehydrogenase (SSADH), α-ketoglutarate decarboxylase (KGDC), glutamate:succinate semialdehyde transaminase, and glutamate decarboxylase, or a combination thereof are as described, above.

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

### Example 1

### Preparation of a Bacterial Strain Deprived of Synthetic Pathway for Lactate and Acetate

### (1) Construction of a Replacement Vector

The L-lactate dehydrogenase (ldh), pyruvate oxidase (poxB), phosphotransacetylase (pta), acetate kinase (ackA), and acetate CoA transferase (actA) genes of Corynebacterium glutamicum (C. glutamicum; CGL) ATCC 13032 were inactivated by homologous recombination. The genes were inactivated by using pK19 mobsacB (ATCC 87098) vector, and the two homologous domains used for recombination were obtained via PCR amplification using the genomic DNA of CGL ATCC 13032 as a template.

The two homologous domains for removing Idh gene was obtained via PCR amplification by using a primer set of IdhA_{—}5′_HindIII (SEQ ID NO: 15) and IdhA_up_{—}3′_XhoI (SEQ ID NO: 16) as an upstream domain of the gene, and a primer set of IdhA_dn_{—}5′_XhoI (SEQ ID NO: 17) and IdhA_{—}3′_EcoRI (SEQ ID NO: 18) as a downstream of the gene, respectively. The PCR amplification was performed by repeating a cycle 30 times, wherein the cycle consisted of denaturation at 95° C. for 30 seconds, annealing at 55° C. for 30 seconds, and extension at 72° C. for 30 seconds. Hereinafter, all PCR amplifications were performed in the same manner. The obtained PCR amplification product was cloned in between HindIII and EcoRI of pK19 mobsacB, and a pK19_Δldh vector was constructed therefrom.

The two homologous domains for removing poxB gene was obtained via PCR amplification by using a primer set of poxB 5′ H3 (SEQ ID NO: 19) and DpoxB_up 3′ (SEQ ID NO:20) as an upstream domain of the gene, and a primer set of DpoxB_dn 5′ (SEQ ID NO:21) and poxB 3′ E1 (SEQ ID NO: 22) as a downstream domain of the gene, respectively. The obtained PCR amplification product was cloned in between HindIII and EcoRI of pK19 mobsacB, and a pK19_ΔpoxB vector was constructed therefrom.

The two homologous domains for removing pta-ackA gene was obtained via PCR amplification by using a primer set of pta 5′ H3 (SEQ ID NO: 23) and Dpta_up_R1 3′ (SEQ ID NO: 24) as an upstream domain of the gene, and a primer set of DackA_dn_R1 5′ (SEQ ID NO: 25) and ackA 3′ Xb (SEQ ID NO: 26) as a downstream domain of the gene, respectively. The obtained PCR amplification product was cloned in between HindIII and XbaI of pK19 mobsacB, and a pK19_Δpta_ackA vector was constructed therefrom.

The two homologous domains for removing actA gene was obtained via PCR amplification by using a primer set of actA 5′ Xb (SEQ ID NO: 27) and DactA_up_R4 3′(SEQ ID NO: 28) as an upstream domain of the gene, and a primer set of DactA_dn_R4 5′ (SEQ ID NO: 29) and actA 3′ H3 (SEQ ID NO: 30) as a downstream domain of the gene, respectively. The obtained PCR amplification product was cloned in between XbaI and HindIII of pK19 mobsacB, and a pK19_ΔactA vector was constructed therefrom.

(2) Preparation of CGL (Δldh, ΔpoxB, Δpta-ackA, ΔactA)

The replacement vectors were introduced together into C. glutamicum ATCC13032 via electroporation. The transformed cells were plated out on an LBHIS agar plate containing 25 μg/mL of kanamycin and incubated at 30° C. The LBHIS agar plate contained 25 g/L of Difco LBTM broth, 18.5 g/L of brain-heart infusion broth, 91 g/L of D-sorbitol and 15 g/L of agar. Hereinafter, all the compositions of LBHIS media are prepared in the same manner. The colonies formed thereon were cultured at 30° C. in BHIS medium (pH 7.0) containing 37 g/L of brain heart infusion powder and 91 g/L of D-sorbitol, and the culture broth was plated out on an LB/Suc10 agar plate, incubated at 30° C., and those with double crossover were selected. The LB/Suc10 agar plate contained 25 g/L of Difco LBTM broth, 15 g/L of agar, and 100 g/L of sucrose.

Genomic DNA was separated from the selected colonies, and the presence of any deletions in the genes was examined. In order to confirm the presence of any deletions in Idh gene, a primer set of IdhA_{—}5′_HindIII and IdhA_{—}3′_EcoRI were used. In order to confirm the presence of any deletions in poxB gene, a PCR was performed by using a primer set of poxB_up_for (SEQ ID NO: 31) and poxB_dn_rev (SEQ ID NO:32). Additionally, in order to confirm the presence of any deletions in pta-ackA gene, a primer set of pta_up_for (SEQ ID NO: 33) and ackA_dn_rev (SEQ ID NO: 34) were used. In order to confirm the presence of any deletions in actA gene, a PCR was performed by using a primer set of actA_up_for (SEQ ID NO: 35) and actA_dn_rev (SEQ ID NO: 36).

### Example 2

### Construction of CGL (Δldh, ΔpoxB, Δpta-ackA, ΔactA, pyc^{P458S})

A variant, (hereinafter, ‘PYC^{P458s}’), in which proline, the 458^{th} amino acid of the pyruvate carboxylase of C. glutamicum ATCC 13032 (SEQ ID NO: 14), is replaced with serine, was prepared.

The variant was prepared by replacing a codon CCG, which encodes proline, the 458^{th} amino acid of PYC amino acid, with a codon TCG via overlap extension PCR.

Two PCR products were obtained from a genomic DNA of CGL ATCC 13032, one PCR product by using a primer set of pyc-F1 (SEQ ID NO: 37) and pyc-R1 (SEQ ID NO: 38), and the other PCR product by using a primer set of pyc-F2 (SEQ ID NO: 39) and pyc-R2 (SEQ ID NO: 40). A third PCR product was obtained by using the obtained two PCR products as a template and a primer set of pyc-F1 and pyc-R2. The third PCR product was cloned into a XbaI restriction site of a pK19mobsacB vector to construct a pK19mobsacB-pyc* vector.

The pK19mobsacB-pyc* vector was introduced into CGL (Δldh, ΔpoxB, Δpta-ackA, ΔactA) of Example 1. A PCR was performed by using a primer set of pyc-F1 and pyc-R2. The sequence of the resulting PCR product was analyzed, and the presence of replacement of pyc gene was examined.

### Example 3

### Preparation of a Bacterial Strain Expressing α-Ketoglutarate Decarboxylase (KGDC) and Succinyl Semialdehyde Dehydrogenase (SSADH)

(1) Construction of pGS_Ptuf::sucA:NCgl0463

pGS_Ptuf::sucA:NCgl0463 vector was constructed as follows in order to express KGDC gene (sucA, SEQ ID NO: 4) of E. coli, and SSADH gene (NCgl0463, SEQ ID NO: 65) of Corynebacterium glutamicum under the tuf promoter.

### 1) Construction of PGS EX4

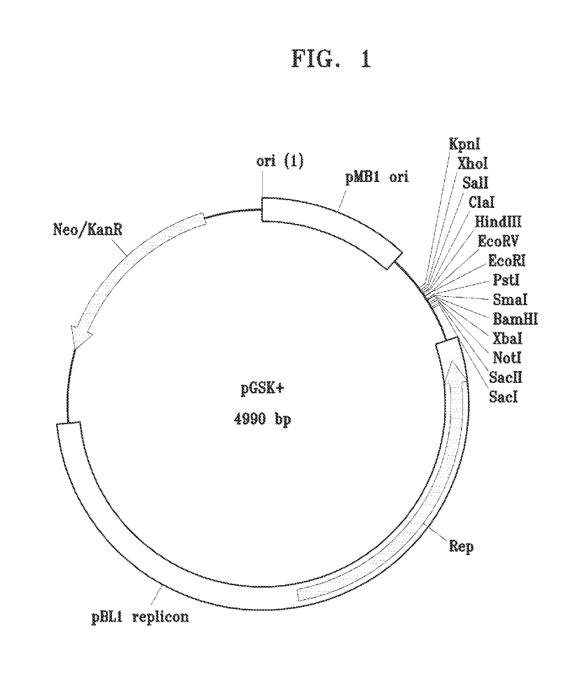

The following four PCR products were obtained by using Phusion High-Fidelity DNA Polymerase (New England Biolabs, cat.# M0530). A PCR was performed by using pET2(GenBank accession number: AJ885178.1), i.e., a vector for screening a promoter of Corynebacterium glutamicum, as a template and by using a primer set of MD-616 (SEQ ID NO: 41) and MD-618 (SEQ ID NO: 42), and a primer set of MD-615 (SEQ ID NO: 43) and MD-617 (SEQ ID NO: 44). In addition, another PCR was performed by using pEGFP-C1 (Clontech) as a template, and by using a primer set of MD-619(SEQ ID NO: 45) and MD-620 (SEQ ID NO: 46), and a primer set of LacZa-NR (SEQ ID NO: 47) and MD-404 (SEQ ID NO: 48). Each of the PCR products, i.e., 3010 bp, 854 bp, 809 bp, and 385 bp fragments, was cloned into a circular plasmid by using an In-Fusion EcoDry PCR Cloning Kit (Clontech, cat.#639690). The cloned vector was transformed into a One Shot TOP10 Chemically Competent Cell (Invitrogen, cat.# C4040-06), cultured in LB medium containing 25 mg/L of kanamycin, and growing colonies were selected. The vector was recovered from the selected colonies, and the nucleotide sequence of the vector was confirmed by analyzing the entire length of the vector sequence. The vector was assigned as pGSK+ (FIG. 1).

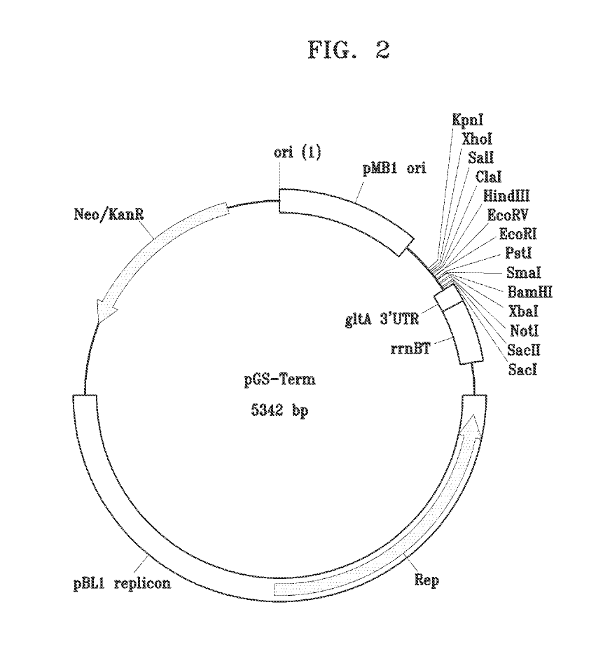

Additionally, 3′UTR of C. glutamicum gltA (NCgl0795) and rho-independent terminator of E. coli rrnB were inserted into the pGSK+ vector as follows. A 108 bp PCR product of gltA 3′UTR was obtained by performing a PCR by using the genomic DNA of C. glutamicum (ATCC13032) as a template, and by using a primer set of MD-627 (SEQ ID NO: 49) and MD-628 (SEQ ID NO: 50). Furthermore, a 292 bp PCR product of rrnB terminator was obtained by using the genomic DNA of E. coli (MG1655) and by using a primer set of MD-629 (SEQ ID NO: 51) and MD-630 (SEQ ID NO: 52). The two amplified fragments were inserted into pGSK+ digested with SacI by using an In-Fusion EcoDry PCR Cloning Kit (Clontech, cat.#639690). The cloned vector was transformed into a One Shot TOP10 Chemically Competent Cell (Invitrogen, cat.# C4040-06), cultured in LB medium containing 25 mg/L of kanamycin, and growing colonies were selected. The vector was recovered from the selected colonies, and the nucleotide sequence of the vector was confirmed by analyzing the entire length of the vector sequence. The vector was assigned as pGS-Term (FIG. 2).

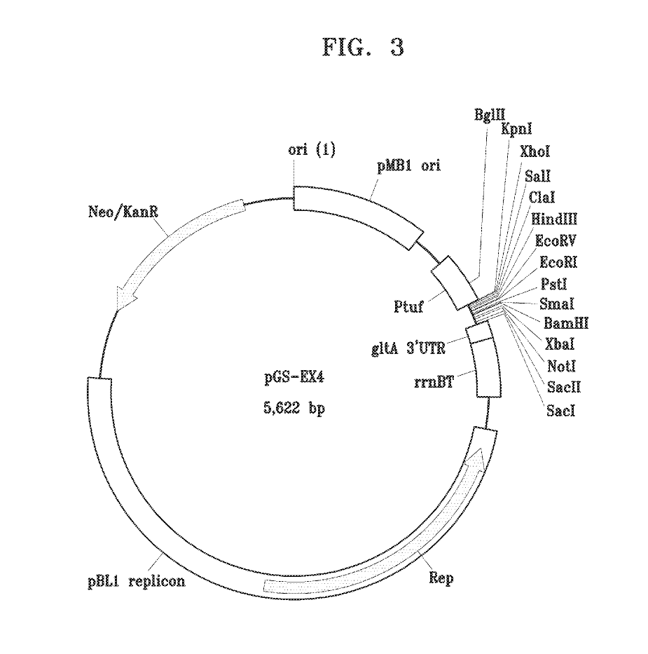

In addition, a Ptuf was obtained by using the genomic DNA of C. glutamicum ATCC 13032 as a template, and by using a primer set of Tuf-F (SEQ ID NO: 53) and Tuf-R (SEQ ID NO: 54). Ptuf is a promoter of tuf gene derived from C. glutamicum. The obtained Ptuf fragment was cloned into the KpnI restriction site of the pGS-Term vector by using an In-Fusion® HD Cloning Kit (Clontech 639648) and pGS_EX4 vector was obtained therefrom (FIG. 3).

2) Construction of PGS Ptuf::sucA:NCgl0463

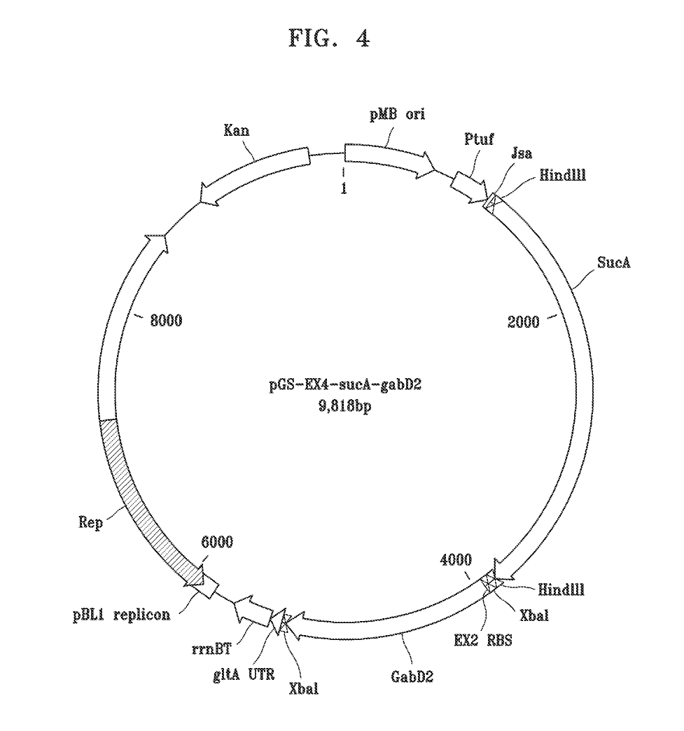

sucA gene was amplified by using the genomic DNA of MG1655, which belong to E. coli K12, as a template and by using a primer set of sucA-F (SEQ ID NO: 55) and sucA-R (SEQ ID NO: 56). Furthermore, the gabD1 (NCgl0463) gene was amplified by using the genomic DNA of C. glutamicum ATCC 13032 as a template, and by using a primer set of NCgl0463_RBS-F (SEQ ID NO: 57) and NCgl0463-R (SEQ ID NO: 58). Each of the amplified products was cloned into a HindIII-XbaI restriction site of a pGS_EX4 vector, and pGS_Ptuf::sucA:NCgl0463 vector was obtained therefrom (FIG. 4). FIG. 4 shows a map of pGS_Ptuf::sucA:NCgl0463 vector.

(2) Construction of CGL (Δldh, ΔpoxB, Δpta-ackA, ΔactA, pyC^{P458S}, pGS_Ptuf::sucA:NCgl0463)

pGS_Ptuf::sucA:NCgl0463 vector was introduced into a C. glutamicum ATCC 13032 (Δldh, ΔpoxB, Δpta-ackA, ΔactA, pyC^{P458}) competent cell via electroporation. The resultant was plated out on LBHIS medium containing 25 μg/mL of kanamycin and incubated at 30° C. CGL (Δldh, ΔpoxB, Δpta-ackA, ΔactA, pyC^{P458S}, pGS_Ptuf::sucA:NCgl0463) was obtained by selecting the strains resistant to kanamycin.

The expressions of sucA and NCgl0463 were examined via RT-PCR by using a primer set of SucA-RTF (SEQ ID NO: 59) and SucA-RTR (SEQ ID NO: 60), and by using a primer set of NCgl0463-RTF (SEQ ID NO: 61) and NCgl0463-RTR (SEQ ID NO: 62). Forty cycles of RT-PCR were performed where each cycle consisted of denaturation at 95° C. for 15 seconds, annealing at 60° C. for 15 seconds, and extension at 72° C. for 30 seconds. As a result, the expressions of sucA and NCgl0463 were confirmed.

### Example 4

### Cultivation of Bacterial Strains Prepared in Example 3 and Production of Succinic Acid

The productivity of CGL (Δldh, ΔpoxB, Δpta-ackA, ΔactA, pyC^{P458S}, pGS_Ptuf::sucA:NCgl0463) on succinic acid was evaluated.

For seed culture, each bacterial strain was streaked on an active plate containing 5 g/L of yeast extract, 10 g/L of beef extract, 10 g/L of polypeptone, 5 g/L of NaCl and 20 g/L of agar, and incubated at 30° C. for 48 hours. A single colony was inoculated to 5 mL of BHIS medium (37 g/L of brain-heart infusion broth, 91 g/L of D-sorbitol, pH 7.0), and cultured at 30° C. until the OD₆₀₀ of the culture broth became 5.0.

1 mL of the thus obtain culture liquid was inoculated into a 250 mL flask containing 25 mL of BHIS medium. After centrifugation of the resulting culture liquid, the supernatant was discarded and microorganisms were collected, which were then washed with CGXII minimal medium. The minimal medium contains 20 g/L of (NH₄)₂SO₄, 5 g/L of urea, 1 g/L of KH₂PO₄, 1 g/L of K₂HPO₄, 0.25 g/L of MgSO₄.7H₂O, 10 mg/L of CaCl₂, 10 mg/L of FeSO₄.7H₂O, 0.1 mg/L of MnSO₄.H₂O, 1 mg/L of ZnSO₄.7H₂O, 0.2 mg/L of CuSO₄.5H₂O, 20 mg/L of NiCl₂.6H₂O, 0.2 mg/L of biotin, 42 g/L of 3-morpholinopropanesulfonic acid (MOPS) and 4% (w/v) of glucose.

Then, the washed cells were added into a 1-well plate containing 20 mL of CGXII minimal medium, which was not sealed but covered on its top, until the O.D. of the cells became 20. The plate was placed in an incubator (STX 40; Liconic instruments), where oxygen concentration is under control, kept at 30° C. for 16 hours while maintaining the oxygen level in contact with the medium at 5%. The concentration of oxygen dissolved in the medium was 1.0 ppm. The sample was collected, centrifuged, and the concentrations of succinic acid and glucose of the resulting supernatant were analyzed via HPLC. Table 1 shows the analytical results.

TABLE 1

| | Glucose | Succinate |
|---|---|---|
| Strain | consumption (g/L) | production (g/L) |
| S006/pGSEx4 | 39.9 ± 0.6 | 0.36 ± 0.08 |
| S006/Ptuf::sucA:gabD1 | 60.7 ± 0.3 | 1.06 ± 0.24 |

In Table 1, S006/pGSEx4 represents a control group strain formed by introducing pGSEx4 to the ATCC 13032 (Δldh, ΔpoxB, Δpta-ackA, ΔactA, pyc^{P458S}) strain, and S006/Ptuf::sucA:gabD1 represents an experimental group strain formed by the Ptuf::sucA:gabD1 vector to the same ATCC 13032 strain.

As shown in Table 1, the bacterial strain introduced with pGS_Ptuf::sucA:NCgl0463 showed about a three-fold increase in the production of succinic acid as compared to the control, a bacterial strain introduced with pGS_EX4. Accordingly, it was confirmed that the productivity of succinic acid of a bacterial cell in a microaerobic condition can be considerably increased by over-expression of KGDC and SSADH.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A genetically engineered bacterial cell, wherein activity of a pathway in the cell of converting α-ketoglutarate (AKG) into succinate semialdehyde (SSA) or activity of succinyl semialdehyde dehydrogenase (SSADH) in the cell is increased as compared to the activity in a parent cell, wherein the pathway of converting AKG into SSA includes α-ketoglutarate decarboxylase (KGDC); glutamate:succinate semialdehyde transaminase and glutamate decarboxylase; or a combination thereof, and the KGDC catalyzes the conversion of α-ketoglutarate into SSA, and the SSADH catalyzes the conversion of SSA into succinic acid.

2. The genetically engineered bacterial cell according to claim 1, wherein the cell produces succinic acid.

3. The genetically engineered bacterial cell according to claim 1, wherein the cell produces succinic acid under microaerobic conditions.

4. The genetically engineered bacterial cell according to claim 1, wherein the cell is a Corynebacterium cell.

5. The genetically engineered bacterial cell according to claim 1, wherein the activity of a pathway in the cell of converting AKG into SSA or activity of succinyl SSADH is increased by an increase in copy number of a gene encoding KGDC, a gene encoding glutamate: succinate semialdehyde transaminase, a gene encoding glutamate decarboxylase, a gene encoding SSADH, or a combination thereof, as compared to a parent cell, or by modification of regulatory sequences for expressing the gene(s).

6. The genetically engineered bacterial cell according to claim 1, wherein the activity of a pathway in the cell of converting AKG into SSA or activity of succinyl SSADH is increased by a mutation in the amino acid sequence of KGDC, glutamate:succinate semialdehyde transaminase, glutamate decarboxylase, SSADH, or a combination thereof.

7. The genetically engineered bacterial cell according to claim 5, wherein the bacterial cell comprises an exogenous gene encoding KGDC, an exogenous gene encoding glutamate:succinate semialdehyde transaminase, an exogenous gene encoding glutamate decarboxylase, an exogenous gene encoding SSADH, or a combination thereof.

8. The genetically engineered bacterial cell according to claim 5, wherein the KGDC is from Corynebacterium., Rhodococcus sp., Mycobacterium sp., or Escherichia sp.

9. The genetically engineered bacterial cell according to claim 5, wherein the succinyl semialdehyde dehydrogenase is from Corynebacterium., Rhodococcus sp., Gordonia sp., Mycobacterium sp., or Escherichia sp.

10. The genetically engineered bacterial cell according to claim 5, wherein the KGDC has an amino acid sequence with a sequence identity of 95% or higher with the amino acid sequence of SEQ ID NO: 1 or the amino acid sequence of SEQ ID NO: 2.

11. The genetically engineered bacterial cell according to claim 5, wherein the succinyl semialdehyde dehydrogenase has an amino acid sequence with a sequence identity of 95% or higher with the amino acid sequence of SEQ ID NO: 5, an amino acid sequence with a sequence identity of 95% or higher with the amino acid sequence of SEQ ID NO: 63, or an amino acid sequence with a sequence identity of 95% or higher with the amino acid sequence of SEQ ID NO: 64.

12. The genetically engineered bacterial cell according to claim 1 comprising an inactivated or attenuated gene encoding succinyl-CoA synthetase.

13. The genetically engineered bacterial cell according to claim 1 comprising an inactivated or attenuated L-lactate dehydrogenase gene, pyruvate oxidase gene, phosphotransacetylase gene, acetate kinase gene, acetate CoA transferase gene, or a combination thereof.

14. The genetically engineered bacterial cell according to claim 1, wherein pyruvate carboxylase activity catalyzing the conversion of pyruvate into oxaloacetate is increased in the genetically engineered bacterial cell as compared to a parent cell.

15. The genetically engineered bacterial cell according to claim 14, wherein the pyruvate carboxylase comprises SEQ ID NO: 14 with amino acid substitution P458S.

16. The genetically engineered bacterial cell according to claim 5, wherein the copy number is increased by amplification of endogenous gene(s).

17. The genetically engineered bacterial cell according to claim 5, wherein the expression of the gene(s) is increased by modification of regulatory sequences for expressing the gene(s).

18. A method for producing succinic acid comprising;
culturing the cell according to claim 1, whereby the cell produces succinic acid; and
recovering succinic acid from the culture.

19. The method according to claim 18, wherein the culturing is carried out under microaerobic conditions.

20. A method of improving succinic acid production by a bacterial cell, the method comprising increasing the activity of succinyl semialdehyde dehydrogenase (SSADH), α-ketoglutarate decarboxylase (KGDC), glutamate:succinate semialdehyde transaminase, and glutamate decarboxylase, or a combination thereof, in the cell.

21. The method of claim 20, wherein the method comprises increasing the copy number of a gene encoding succinyl semialdehyde dehydrogenase (SSADH), α-ketoglutarate decarboxylase (KGDC), glutamate:succinate semialdehyde transaminase and glutamate decarboxylase, or a combination thereof, in the cell.

22. The genetically engineered cell of claim 12, wherein the gene encoding succinyl-CoA synthetase comprises an amino acid sequence with 95% or higher identity with the amino acid sequence of SEQ ID NO: 7 or the amino acid sequence of SEQ ID NO: 8.


SEQUENCE LISTING (69 sequences)

SEQ ID NO: 1 - protein, 543 aa, Corynebacterium glutamicum
MSSTPAQDLARAVIDSLAPHVTDVVLCPGSRNSPLSLELLARQDLRVHVRIDERSASFLALSLARTQARPVAVVMTSGTAVANCLPAVAEAAHAHIPLIVLSADRPAHLVGTGASQTINQTGIFGDLAPTVGITELDQVAQIAESLAQGASQIPRHFNLALDVPLVAPELPELHGEAVGASWTHRWINHGEVTVDLGEHTLVIAGDEAWEVEGLEDVPTIAEPTAPKPYNPVHPLAAEILLKEQVSAEGYVVNTRPDHVIVVGHPTLHRGVLKLMSDPGIKLTVLSRTDIITDPGRHADQVGSTVKVTGTQEKQWLKICSAASELAADGVRDVLDNQEFGFTGLHVAAAVADTLGTGDTLFAAASNSIRDLSLVGMPFDGVDTFSPRGVAGIDGSVAQAIGTSLAVQSRHPDEIRAPRTVALLGDLSFLHDIGGLLIGPDEPRPENLTIVVSNDNGGGIFELLETGADGLRPNFERAFGTPHDASIADLCAGYGIEHQVVDNLQDLIIALVDTTEVSGFTIIEASTVRDTRRAQQQALMDTVH

SEQ ID NO: 2 - protein, 933 aa, Escherichia coli
MQNSALKAWLDSSYLSGANQSWIEQLYEDFLTDPDSVDANWRSTFQQLPGTGVKPDQFHSQTREYFRRLAKDASRYSSTISDPDTNVKQVKVLQLINAYRFRGHQHANLDPLGLWQQDKVADLDPSFHDLTEADFQETFNVGSFASGKETMKLGELLEALKQTYCGPIGAEYMHITSTEEKRWIQQRIESGRATFNSEEKKRFLSELTAAEGLERYLGAKFPGAKRFSLEGGDALIPMLKEMIRHAGNSGTREVVLGMAHRGRLNVLVNVLGKKPQDLFDEFAGKHKEHLGTGDVKYHMGFSSDFQTDGGLVHLALAFNPSHLEIVSPVVIGSVRARLDRLDEPSSNKVLPITIHGDAAVTGQGVVQETLNMSKARGYEVGGTVRIVINNQVGFTTSNPLDARSTPYCTDIGKMVQAPIFHVNADDPEAVAFVTRLALDFRNTFKRDVFIDLVCYRRHGHNEADEPSATQPLMYQKIKKHPTPRKIYADKLEQEKVATLEDATEMVNLYRDALDAGDCVVAEWRPMNMHSFTWSPYLNHEWDEEYPNKVEMKRLQELAKRISTVPEAVEMQSRVAKIYGDRQAMAAGEKLFDWGGAENLAYATLVDEGIPVRLSGEDSGRGTFFHRHAVIHNQSNGSTYTPLQHIHNGQGAFRVWDSVLSEEAVLAFEYGYATAEPRTLTIWEAQFGDFANGAQVVIDQFISSGEQKWGRMCGLVMLLPHGYEGQGPEHSSARLERYLQLCAEQNMQVCVPSTPAQVYHMLRRQALRGMRRPLVVMSPKSLLRHPLAVSSLEELANGTFLPAIGEIDELDPKGVKRVVMCSGKVYYDLLEQRRKNNQHDVAIVRIEQLYPFPHKAMQEVLQQFAHVKDFVWCQEEPLNQGAWYCSQHHFREVIPFGASLRYAGRPASASPAVGYMSVHQKQQQDLVNDALNVE

SEQ ID NO: 3 - DNA, 1632 bp, Corynebacterium glutamicum
atgtccagcacgccagctcaagatcttgcccgcgccgttattgattccctcgcaccacacgtcactgacgtggtgttatgcccaggatccaggaactcaccgttgtcgcttgagttgctggcgcggcaggatctgcgtgtccatgtgcgtatcgacgagcgcagcgcctcatttttggcgctgtccctagcgcgtacccaggcccggccggtggctgtggtgatgacctccggcacggctgtagctaactgcctgcctgctgttgctgaagctgcgcatgcccatatcccgttgattgtgctctctgctgaccgtcctgcacatttggtgggaacgggggcgagccaaacgattaaccagaccggtatttttggtgatcttgcaccgacggtcggtatcactgagctggatcaggtagcgcagattgctgaaagccttgctcagggggcttcccagattccgcgtcatttcaatcttgcacttgatgttcctttggttgctcctgaactgccagagcttcatggtgaggcagttggagcatcatggacgcatcgctggatcaaccacggtgaggtgaccgtggacctgggggagcacaccctcgtgattgccggtgatgaagcatgggaagtggaagggctggaagatgtgcccaccatcgctgaacctactgcaccaaagccttataatccggtgcacccactggctgctgaaatcttgctgaaggagcaggtctccgcggaaggctatgtggtaaacaccaggcctgatcatgtgatcgtggtgggacaccccacgctgcaccgcggagtgttgaagttgatgtcagatcctggcattaaattaactgtgctttcacgcaccgatatcatcactgatcccggccgccatgccgatcaggtgggcagcacagtgaaagtcaccggcacccaggaaaagcagtggctaaagatctgttcggcagcatcagaacttgcggccgatggtgtgcgtgacgtcctggacaaccaagaattcggtttcaccggcctccatgttgccgcagccgtggcggataccttaggcaccggcgatactctctttgctgcagcatccaactcaatccgtgacctctccctggtgggtatgccttttgatggcgtggataccttctccccacgaggtgtcgcaggcattgatggttctgttgctcaagcaatcggcacttcacttgctgtgcagtcccgccaccccgatgaaatccgcgcgccacgcactgtggcccttctgggcgatctgtcgttccttcacgatattggcggactgctcatcggccctgatgaaccacgcccagaaaacctcaccatcgtggtctccaacgacaacggtggcggaatcttcgaactcctagaaaccggcgcagatggtctccgccccaacttcgagcgtgctttcggtaccccacacgacgcgtccatcgcggatctctgcgcaggctacggcattgaacaccaagtggtagacaacctccaagacctcatcatcgcgctagttgataccaccgaagtatccggattcaccattattgaagcttcgaccgtccgagatacccgccgtgcacaacagcaagctctcatggacacggtgcactaa

SEQ ID NO: 4 - DNA, 2802 bp, Escherichia coli
atgcagaacagcgctttgaaagcctggttggactcttcttacctctctggcgcaaaccagagctggatagaacagctctatgaagacttcttaaccgatcctgactcggttgacgctaactggcgttcgacgttccagcagttacctggtacgggagtcaaaccggatcaattccactctcaaacgcgtgaatatttccgccgcctggcgaaagacgcttcacgttactcttcaacgatctccgaccctgacaccaatgtgaagcaggttaaagtcctgcagctcattaacgcataccgcttccgtggtcaccagcatgcgaatctcgatccgctgggactgtggcagcaagataaagtggccgatctggatccgtctttccacgatctgaccgaagcagacttccaggagaccttcaacgtcggttcatttgccagcggcaaagaaaccatgaaactcggcgaactgctggaagcactcaagcaaacctactgcggcccgattggtgccgagtatatgcacattaccagcactgaagaaaaacgctggatccaacagcgtattgagtctggtcgcgcgactttcaatagcgaagagaaaaaacgcttcttaagcgaactgaccgccgctgaaggccttgaacgttacctcggcgcaaaattccctggcgcaaaacgcttctcgctggaaggcggtgacgcgttaatcccgatgcttaaagagatgatccgccacgctggcaacagcggcacccgcgaagtggttctcggaatggcgcaccgtggtcgtctgaacgtgctggtgaacgtgctgggtaaaaaaccgcaagacttgttcgacgagtttgccggtaaacataaagaacacctcggcacgggcgacgtgaaataccacatgggcttctcgtctgacttccagaccgatggcggcctggttcacctggcgctggcgtttaacccgtctcaccttgagattgtaagcccggtcgttatcggttctgttcgtgcccgtctggacagacttgatgagccgagcagcaacaaagtgctgccaatcaccattcatggtgacgccgcagtgaccgggcagggcgtggttcaggaaaccctgaacatgtcgaaagcgcgtggttatgaagttggcggtacggtacgtatcgttatcaacaaccaggttggcttcaccacctctaacccgctggatgcccgttcgacgccgtactgtactgatatcggtaagatggttcaggcaccgattttccacgttaacgcggatgatccggaagccgttgcctttgttacccgtctggcgctcgatttccgtaacacctttaaacgtgatgtcttcatcgacctggtatgctaccgccgtcacggccacaacgaagccgacgagccgagcgcaacccagccgctgatgtatcagaaaatcaaaaaacatccgacgccgcgcaaaatctacgctgacaagctggagcaggaaaaagtcgcgacgctggaagatgccaccgagatggttaacctgtaccgcgatgcgctggatgctggcgattgcgttgtagcagagtggcgtccgatgaacatgcactctttcacctggtcgccgtacctcaaccatgaatgggacgaagagtacccgaacaaagttgagatgaagcgcctgcaggaactggctaaacgcatcagcacggtgccggaagcggttgaaatgcagtctcgcgttgccaagatttatggcgatcgccaggcgatggcagccggtgagaaactgttcgactggggcggcgcggaaaacctcgcttacgccacgttggttgacgaaggcattccggttcgcctgtcgggtgaagactccggtcgcggtaccttcttccaccgccacgcggtgatccacaaccagtctaacggttccacttacacgccgctgcaacatatccataacggccagggcgcgttccgcgtctgggactctgtactgtctgaagaagccgtactggcgtttgaatacggttatgccaccgcagaaccacgcaccctgaccatctgggaagcacagttcggtgacttcgccaacggtgcacaggtggttatcgaccagttcatctcctctggcgaacagaaatggggccggatgtgtggcctggtgatgttgctgccgcacggttacgaagggcaggggccggagcactcctccgcgcgtctggaacgttatctgcaactttgcgctgagcaaaacatgcaggtttgcgtaccttctaccccggcacaggtttaccacatgctgcgtcgtcaggcgttgcgcgggatgcgtcgtccactggtcgtgatgtcgccgaaatccctgctgcgtcatccgctggcggtatccagcctcgaagaactggcgaacggcaccttcctgccagccatcggtgaaatcgacgagcttgatccgaagggcgtgaagcgcgtagtgatgtgttctggtaaggtttattacgacctgctggaacaacgtcgtaagaacaatcaacacgatgtcgccattgtgcgtatcgagcaactctacccgttcccgcataaagcgatgcaggaagtgttgcagcagtttgctcacgtcaaggattttgtctggtgccaggaagagccgctcaaccagggcgcatggtactgcagccagcatcatttccgtgaagtgattccgtttggggcttctctgcgttatgcaggccgcccagcctccgcctctccggcggtagggtatatgtccgttcaccagaaacagcaacaagatctggttaatgacgcgctgaacgtcgaataa

SEQ ID NO: 5 - protein, 453 aa, Corynebacterium glutamicum
MSLTFPVINPSDGSTITELENHDSTQWMSALSDAVAAGPSWAAKTPRERSVVLTAIFEALTERAQELAEIIHLEAGKSVAEALGEVAYGAEYFRWFAEEAVRLPGRYGQSPSGIGHIAVTRAPVGPVLAITPWNFPIAMATRKIAPALAAGCPVLVKPASETPLTMVKVGEIIASVFDTFNIPQGLVSIITTTRDAELSAELMADPRLAKVTFTGSTNVGRILVRQSADRLLRTSMELGGNAAFVIDEAADLDEAVSGAIAAKLRNAGQVCIAANRFLVHESRAAEFTSKLATAMQNTPIGPVISARQRDRIAALVDEAITDGARLIIGGEVPDGSGFFYPATILADVPAQSRIVHEEIFGPVATIATFTDLAEGVAQANSTEFGLAAYGFSNNVKATQYMAEHLEAGMVGINRGAISDPAAPFGGIGQSGFGREGGTEGIEEYLSVRYLALP

SEQ ID NO: 6 - DNA, 1362 bp, Corynebacterium glutamicum
gtgtctttgaccttcccagtaatcaaccccagcgatggctccaccatcaccgagctagaaaaccacgattccacccagtggatgtccgcgctctctgatgcagttgcagctggtccttcatgggctgcgaaaactccccgcgaaagatccgtggtactcaccgcaatcttcgaagcactgaccgaacgcgcccaagaacttgcagagatcatccacctggaagctggaaaatccgttgcagaagctcttggtgaagtcgcttatggtgcagaatacttccgttggtttgcggaagaagcagtgcgcctgcccggccgctacggacagtcaccttccggaatcggtcacatcgccgtcacccgcgcacccgtgggaccagtgctggcgatcaccccatggaatttccccatcgccatggccacccgcaaaatcgccccagccctggccgctggttgccccgtgttggtgaaacctgcttccgaaaccccactgaccatggtcaaagtgggggagatcatcgcctccgtctttgatacctttaatatcccgcagggcttggtctcaatcatcaccaccactcgagatgcagagctatcggcagaactcatggctgatcctcgcttggctaaagtcaccttcactggatcaaccaacgtgggacgcatcctggtccgccaatccgcggaccgactgctgcgcacctccatggaactcggcggaaatgcagcttttgttatcgacgaagccgcagacctcgacgaagccgtatccggtgccatcgccgcaaaactccgcaacgccggccaagtatgcatcgcagctaaccgtttcttggttcatgaatcccgcgctgccgaattcacctcaaagctggcgacagccatgcagaacactcccattgggccggtgatttctgcccgccaacgcgaccggatcgcagcactagtggatgaagccatcaccgacggcgcccgcctcatcatcggtggggaggtccccgacggctccggcttcttctatccagccaccatcttggccgatgtccctgcacagtcacggattgtgcatgaggaaatcttcggacctgtggccaccattgccactttcaccgacttggccgaaggcgttgcacaagcaaattccaccgaattcggcctcgcagcctacggattcagcaacaatgtgaaagcaacacagtacatggcggaacacttggaagccggaatggtcggaatcaacagaggcgccatctctgacccagcagcaccttttggcggcatcggacaatccggcttcggcagagaaggcggaaccgaaggaatcgaagaatatctctccgtgcgttacctcgctttgccgtga

SEQ ID NO: 7 - protein, 294 aa, Corynebacterium glutamicum
MSIFLNSDSRIIIQGITGSEGSEHARRILASGAKLVGGTNPRKAGQTILINDTELPVFGTVKEAMEETGADVTVIFVPPAFAKAAIIEAIDAHIPLCVIITEGIPVRDASEAWAYAKKVGHTRIIGPNCPGIITPGESLAGITPANIAGSGPIGLISKSGTLTYQMMYELSDIGISTAIGIGGDPIIGTTHIDALEAFEADPETKAIVMIGEIGGDAEERAADFISKHVTKPVVGYVAGFTAPEGKTMGHAGAIVTGSEGTARAKKHALEAVGVRVGTTPSETAKLMREVVAAL

SEQ ID NO: 8 - protein, 398 aa, Corynebacterium glutamicum
MDLFEYQARDLFETHGVPVLKGIVASTPEAARKAAEEIGGLTVVKAQVKVGGRGKAGGVRVAPTSAQAFDAADAILGMDIKGHTVNQVMVAQGADIAEEYYFSILLDRANRSYLAMCSVEGGMEIEILAKEKPEALAKVEVDPLTGIDEDKAREIVTAAGFETEVAEKVIPVLIKIWQVYYEEEATLVEVNPLVLTDDGDVIALDGKITLDDNADFRHDNRGALAESAGGLDILELKAKKNDLNYVKLDGSVGIIGNGAGLVMSTLDIVAAAGERHGGQRPANFLDIGGGASAESMAAGLDVILGDSQVRSVFVNVFGGITACDVVAKGIVGALDVLGDQATKPLVVRLDGNNVVEGRRILAEYNHPLVTVVEGMDAAADHAAHLANLAQHGQFATAN

SEQ ID NO: 9 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 10 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 11 - protein, 461 aa, Corynebacterium glutamicum
MSDTPTSALITTVNRSFDGFDLEEVAADLGVRLTYLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 12 - protein, 397 aa, Corynebacterium glutamicum
MALALVLNSGSSSIKFQLVNPENSAIDEPYVSGLVEQIGEPNGRIVLKIEGEKYTLETPIADHSEGLNLAFDLMDQHNCGPSQLEITAVGHRVVHGGILFSAPELITDEIVEMIRDLIPLAPLHNPANVDGIDVARKILPDVPHVAVFDTGFFHSLPPAAALYAINKDVAAEHGIRRYGFHGTSHEFVSKRVVEILEKPTEDINTITFHLGNGASMAAVQGGRAVDTSMGMTPLAGLVMGTRSGDIDPGIVFHLSRTAGMSIDEIDNLLNKKSGVKGLSGVNDFRELREMIDNNDQDAWSAYNIYIHQLRRYLGSYMVALGRVDTIVFTAGVGENAQFVREDALAGLEMYGIEIDPERNALPNDGPRLISTDASKVKVFVIPTNEELAIARYAVKFA

SEQ ID NO: 13 - protein, 250 aa, Corynebacterium glutamicum
MSHMINKSISSTAEAVADIPDGASIAVGGFGLVGIPTALILALREQGAGDLTIISNNLGTDGFGLGLLLLDKKISKSIGSYLGSNKEYARQYLEGELTVEFTPQGTLAERLRAGGAGIPAFYTTAGVGTQVAEGGLPQRYNTDGTVAVVSQPKETREFNGQLYVMEEGIRADYALVHAHKADRFGNLVFRKTAQNFNPDAAMSGKITIAQVEHFVDELHPDEIDLPGIYVNRVVHVGPQETGIENRTVSN

SEQ ID NO: 14 - protein, 1140 aa, Corynebacterium glutamicum
MSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 15 - DNA, 43 bp, Artificial Sequence
  Synthetic (ldhA_5′_HindIII)
catgattacgccaagcttgagagcccaccacattgcgatttcc

SEQ ID NO: 16 - DNA, 42 bp, Artificial Sequence
  Synthetic (ldhA_up_3′_XhoI)
tcgaaactcgagtttcgatcccacttcctgatttccctaacc

SEQ ID NO: 17 - DNA, 39 bp, Artificial Sequence
  Synthetic (ldhA_dn_5′_XhoI)
tcgaaactcgagtaaatctttggcgcctagttggcgacg

SEQ ID NO: 18 - DNA, 46 bp, Artificial Sequence
  Synthetic (ldhA_3′_EcoRI)
acgacggccagtgaattcgacgacatctgagggtggataaagtggg

SEQ ID NO: 19 - DNA, 42 bp, Artificial Sequence
  Synthetic (poxB 5′ H3)
catgattacgccaagctttcagcgtgggtcgggttctttgag

SEQ ID NO: 20 - DNA, 32 bp, Artificial Sequence
  Synthetic (DpoxB_up 3′)
aatcatcatctgaactcctcaacgttatggct

SEQ ID NO: 21 - DNA, 37 bp, Artificial Sequence
  Synthetic (DpoxB_dn 5′)
ggagttcagatgatgattgatacacctgctgttctca

SEQ ID NO: 22 - DNA, 44 bp, Artificial Sequence
  Synthetic (poxB 3′ E1)
acgacggccagtgaattcatgtcccgaatccacttcaatcagag

SEQ ID NO: 23 - DNA, 44 bp, Artificial Sequence
  Synthetic (pta 5′ H3)
catgattacgccaagcttccctccatgatacgtggtaagtgcag

SEQ ID NO: 24 - DNA, 43 bp, Artificial Sequence
  Synthetic (Dpta_up_R1 3′)
gttccctgttaatgtaaccagctgaggtcggtgtgtcagacat

SEQ ID NO: 25 - DNA, 48 bp, Artificial Sequence
  Synthetic (DackA_dn_R1 5′)
ttacattaacagggaaccggaagagttagctatcgctaggtacgcggt

SEQ ID NO: 26 - DNA, 40 bp, Artificial Sequence
  Synthetic (ackA 3′ Xb)
acccggggatcctctagagggctgatgtgatttctgcggg

SEQ ID NO: 27 - DNA, 41 bp, Artificial Sequence
  Synthetic (actA 5′ Xb)
ggtggcggccgctctagaggtctgagctttattcctgggct

SEQ ID NO: 28 - DNA, 36 bp, Artificial Sequence
  Synthetic (DactA_up_R4 3′)
tctggatagaagcatctaagccagcgccggtgaagc

SEQ ID NO: 29 - DNA, 46 bp, Artificial Sequence
  Synthetic (DactA_dn_R4 5′)
agatgcttctatccagagctccggtgacaacaagtacatgcagacc

SEQ ID NO: 30 - DNA, 39 bp, Artificial Sequence
  Synthetic (actA 3′ H3)
gacggtatcgataagcttcgtacgatgcttgagcggtat

SEQ ID NO: 31 - DNA, 19 bp, Artificial Sequence
  Synthetic (poxB_up_for)
ggctgaaaccaaaccagac

SEQ ID NO: 32 - DNA, 22 bp, Artificial Sequence
  Synthetic (poxB_dn_rev)
ctgcatgatcggttagatacag

SEQ ID NO: 33 - DNA, 18 bp, Artificial Sequence
  Synthetic (pta_up_for)
gcgtggaattgagatcgg

SEQ ID NO: 34 - DNA, 18 bp, Artificial Sequence
  Synthetic (ackA_dn_rev)
cagagcgatttgtggtgg

SEQ ID NO: 35 - DNA, 20 bp, Artificial Sequence
  Synthetic (actA_up_for)
tgaagcaatggtgtgaactg

SEQ ID NO: 36 - DNA, 19 bp, Artificial Sequence
  Synthetic (actA_dn_rev)
gctaccaaacactagcctg

SEQ ID NO: 37 - DNA, 25 bp, Artificial Sequence
  Synthetic (pyc-F1)
gctctagattgagcacaccgtgact

SEQ ID NO: 38 - DNA, 19 bp, Artificial Sequence
  Synthetic (pyc-R1)
ctgaaggaggtgcgagtga

SEQ ID NO: 39 - DNA, 19 bp, Artificial Sequence
  Synthetic (pyc-F2)
tcactcgcacctccttcag

SEQ ID NO: 40 - DNA, 31 bp, Artificial Sequence
  Synthetic (pyc-R2)
gctctagagaagcagcatctgaatgtttaca

SEQ ID NO: 41 - DNA, 46 bp, Artificial Sequence
  Synthetic (MD-616)
aaagtgtaaagcctgggaacaacaagacccatcatagtttgccccc

SEQ ID NO: 42 - DNA, 36 bp, Artificial Sequence
  Synthetic (MD-618)
gttcttctaatcagaattggttaattggttgtaaca

SEQ ID NO: 43 - DNA, 40 bp, Artificial Sequence
  Synthetic (MD-615)
gcgtaatagcgaagaggggcgtttttccataggctccgcc

SEQ ID NO: 44 - DNA, 40 bp, Artificial Sequence
  Synthetic (MD-617)
gttcaatcataacaccccttgtattactgtttatgtaagc

SEQ ID NO: 45 - DNA, 31 bp, Artificial Sequence
  Synthetic (MD-619)
gggtgttatgattgaacaagatggattgcac

SEQ ID NO: 46 - DNA, 39 bp, Artificial Sequence
  Synthetic (MD-620)
attctgattagaagaactcgtcaagaaggcgatagaagg

SEQ ID NO: 47 - DNA, 17 bp, Artificial Sequence
  Synthetic (LacZa-NR)
cctcttcgctattacgc

SEQ ID NO: 48 - DNA, 21 bp, Artificial Sequence
  Synthetic (MD-404)
cccaggctttacactttatgc

SEQ ID NO: 49 - DNA, 47 bp, Artificial Sequence
  Synthetic (MD-627)
gccaccgcggtggagctcatttagcggatgattctcgttcaacttcg

SEQ ID NO: 50 - DNA, 32 bp, Artificial Sequence
  Synthetic (MD-628)
ttttatttgcaaaaacggccgaaaccatccct

SEQ ID NO: 51 - DNA, 40 bp, Artificial Sequence
  Synthetic (MD-629)
ccgtttttgcaaataaaacgaaaggctcagtcgaaagact

SEQ ID NO: 52 - DNA, 43 bp, Artificial Sequence
  Synthetic (MD-630)
gaacaaaagctggagctaccgtatctgtggggggatggcttgt

SEQ ID NO: 53 - DNA, 36 bp, Artificial Sequence
  Synthetic (Tuf-F)
ctatagggcgaattgggatcacagtaggcgcgtagg

SEQ ID NO: 54 - DNA, 56 bp, Artificial Sequence
  Synthetic (Tuf-R)
gacctcgagggggggcccggtaccggttgtcctcctttgggtggctacgactttcg

SEQ ID NO: 55 - DNA, 26 bp, Artificial Sequence
  Synthetic (sucA-F)
cccaagcttatgcagaacagcgcttt

SEQ ID NO: 56 - DNA, 26 bp, Artificial Sequence
  Synthetic (sucA-R)
cccaagcttttattcgacgttcagcg

SEQ ID NO: 57 - DNA, 47 bp, Artificial Sequence
  Synthetic (gabD2_RBS-F)
tagttctagactcgataagagaggacaacggtgtctttgaccttccc

SEQ ID NO: 58 - DNA, 24 bp, Artificial Sequence
  Synthetic (gabD2-R)
cgctctagatcacggcaaagcgag

SEQ ID NO: 59 - DNA, 19 bp, Artificial Sequence
  Synthetic (SucA-RTF)
gacaccaatgtgaagcagg

SEQ ID NO: 60 - DNA, 18 bp, Artificial Sequence
  Synthetic (SucA-RTR)
tcccagcggatcgagatt

SEQ ID NO: 61 - DNA, 20 bp, Artificial Sequence
  Synthetic (gabD2-RTF)
ccttcccagtaatcaacccc

SEQ ID NO: 62 - DNA, 20 bp, Artificial Sequence
  Synthetic (gabD2-RTR)
caccataagcgacttcacca

SEQ ID NO: 63 - protein, 490 aa, Escherichia coli gabD1
MTINVSELLAKVPTGLLIGDSWVEASDGGTFDVENPATGETIATLASATSEDALAALDAACAVQAEWARMPARERSNILRRGFELVAERAEEFATLMTLEMGKPLAEARGEVTYGNEFLRWFSEEAVRLYGRYGTTPEGNLRMLTALKPVGPCLLITPWNFPLAMATRKVAPAIAAGCVMVLKPARLTPLTSQYFAQTMLDAGLPAGVLNVVSGASASAISNPIMEDDRLRKVSFTGSTPVGQQLLKKAADKVLRTSMELGGNAPFIVFEDADLDLAIEGAMGAKMRNIGEACTAANRFLVHESVADEFGRRFAARLEEQVLGNGLDEGVTVGPLVEEKARDSVASLVDAAVAEGATVLTGGKAGTGAGYFYEPTVLTGVSTDAAILNEEIFGPVAPIVTFQTEEEALRLANSTEYGLASYVFTQDTSRIFRVSDGLEFGLVGVNSGVISNAAAPFGGVKQSGMGREGGLEGIEEYTSVQYIGIRDPYAG

SEQ ID NO: 64 - protein, 521 aa, Escherichia coli gabD3
MIKRLPLGPLPKELHQTLLDLTANAQDAAKVEVIAPFTGETLGFVFDGDEQDVEHAFALSRAAQKKWVHTTAVERKKIFLKFHDLVLKNRELLMDIVQLETGKNRASAADEVLDVAITTRFYANNAGKFLNDKKRPGALPIITKNTQQYVPKGVVGQITPWNYPLTLGVSDAVPALLAGNAVVAKPDLATPFSCLIMVHLLIEAGLPRDLMQVVTGPGDIVGGAIAAQCDFLMFTGSTATGRILGRTMGERLVGFSAELGGKNPLIVAKDADLDKVEAELPQACFSNSGQLCVSTERIYVEEDVYEEVIARFSKAAKAMSIGAGFEWKYEMGSLINQAQLDRVSTFVDQAKAAGATVLCGGKSRPDIGPFFYEPTVLADVPEGTPLLTEEVFGPVVFIEKVATLEEAVDKANGTPYGLNASVFGSSETGNLVAGQLEAGGIGINDGYAATWASVSTPLGGMKQSGLGHRHGAEGITKYAEIRNIAEQRWMSMRGPAKMPRKVYSDTVATALKLGKIFKVLP

SEQ ID NO: 65 - DNA, 1473 bp, Escherichia coli gabD1
atgactattaatgtctccgaactacttgccaaagtccccacgggtctactgattggtgattcctgggtggaagcatccgacggcggtactttcgatgtggaaaacccagcgacgggtgaaacaatcgcaacgctcgcgtctgctacttccgaggatgcactggctgctcttgatgctgcatgcgctgttcaggccgagtgggctaggatgccagcgcgcgagcgttctaatattttacgccgcggttttgagctcgtagcagaacgtgcagaagagttcgccaccctcatgaccttggaaatgggcaagcctttggctgaagctcgcggcgaagtcacctacggcaacgaattcctgcgctggttctctgaggaagcagttcgtctgtatggccgttacggaaccacaccagaaggcaacttgcggatgctgaccgccctcaagccagttggcccgtgcctcctgatcaccccatggaacttcccactagcaatggctacccgcaaggtcgcacctgcgatcgctgcaggttgtgtcatggtgctcaagccagctcgacttaccccgctgacctcccagtattttgctcagaccatgcttgatgccggtcttccagcaggtgtcctcaatgtggtctccggtgcttccgcctctgcgatttccaacccgattatggaagacgatcgccttcgtaaagtctccttcaccggctccaccccagttggccagcagctgctcaaaaaggctgccgataaagttctgcgcacctccatggaacttggtggcaacgcacctttcattgtcttcgaggacgccgacctagatctcgcgatcgaaggtgccatgggtgccaaaatgcgcaacatcggcgaagcttgcaccgcagccaaccgtttcttagtccacgaatccgtcgccgatgaattcggccgtcgcttcgctgcccgccttgaagagcaagtcctaggcaacggcctcgacgaaggcgtcaccgtgggccccctggttgaggaaaaagcacgagacagcgttgcatcgcttgtcgacgccgccgtcgccgaaggtgccaccgtcctcaccggcggcaaggccggcacaggtgcaggctacttctacgaaccaacggtgctcacgggagtttcaacagatgcggctatcctgaacgaagagatcttcggtcccgtcgcaccgatcgtcaccttccaaaccgaggaagaagccctgcgtctagccaactccaccgaatacggactggcctcctatgtgttcacccaggacacctcacgtattttccgcgtctccgatggtctcgagttcggcctagtgggcgtcaattccggtgtcatctctaacgctgctgcaccttttggtggcgtaaaacaatccggaatgggccgcgaaggtggtctcgaaggaatcgaggagtacacctccgtgcagtacatcggtatccgggatccttacgccggctag

SEQ ID NO: 66 - DNA, 1566 bp, Escherichia coli gabD3
atgatcaaacgtcttcctttaggtccgctgcctaaagaacttcatcagactctgcttgatctgaccgcaaatgcccaagatgcggcgaaagtggaggttatagcgccatttactggcgagaccctcggatttgtttttgatggtgatgagcaagacgtcgagcatgcttttgcactttcaagggcagcccagaaaaagtgggtgcacaccacggcagtggaacggaagaagatcttcctgaagtttcatgatctggtattgaaaaaccgtgagctgctcatggacatcgtgcagttggaaacaggcaaaaatcgagcatcggctgccgatgaggtgttggacgttgcgatcaccacccgcttctacgcaaacaatgcaggaaagtttttaaatgacaagaaacgccccggcgcgcttccgatcatcacgaaaaacacacaacagtatgtgcccaagggagtggtcgggcagatcacgccgtggaattaccctttaactttgggagtatctgatgctgttccggcgctgctggcaggaaacgcagtggtggctaaacctgacctcgcgacacctttctcctgcttgatcatggtgcacctgctcattgaagccggtctgccgcgtgatttgatgcaggttgtcaccggccctggcgatattgttggcggtgcgattgcagctcagtgtgatttcctcatgttcactggatccacggccacgggccggatcttgggtcggacaatgggtgagcgtttggtgggtttctctgcggaattaggcggaaagaaccctcttattgtggccaaggatgcagatctggacaaggtggaagctgagcttccgcaggcgtgtttttccaactcggggcaattgtgtgtctccactgaacgtatttatgtcgaggaagacgtgtacgaggaggtgattgcacggtttagcaaggcggcgaaagccatgtccattggtgccggatttgagtggaaatatgagatgggttcgttgatcaatcaggcgcagctggatcgggtgagcacctttgttgatcaggctaaagctgcgggcgccacggtgctgtgcggtggcaagtcacgccctgatattggtcccttcttctatgagcccacggtattggcggatgtcccagagggcaccccactgctcacggaggaagtcttcgggccggtggtgttcatcgaaaaggtagccacactggaagaagccgtcgataaggcaaatggcacgccctacggcctgaatgcgtccgtctttgggtcgtcggaaaccggcaatcttgttgcaggccagctggaagctggcggtatcggtattaatgatggctacgccgcgacgtgggcgagcgtgtccacgcctctgggtggcatgaagcagtcggggctggggcaccgccatggtgcggagggaattacaaaatatgcggagatccgaaacatcgcggagcagcgctggatgtctatgcgtgggccggccaaaatgccgcgaaaggtgtactcagacaccgtggccacagcgctaaagctgggcaaaatctttaaagttttgccgtag

SEQ ID NO: 67 - protein, 426 aa, Escherichia coli gabT
MNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 68 - protein, 466 aa, Escherichia coli Glutamate decarboxylase alpha
MDQKLLTDFRSELLDSRFGAKAISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 69 - protein, 466 aa, Escherichia coli Glutamate decarboxylase beta (gadB)
MDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT